(19) United States
(12) Patent Application Publication
(10) Pub. No.: US 2008/0275351 A1
(43) Pub. Date: Nov. 6, 2008

(54) Model-based pulse wave velocity measurement method

(75) Inventors: Klaus J. Kirchberg, Princeton, NJ (US); Christine H. Lorenz, Frederick, MD (US)

Correspondence Address: SIEMENS CORPORATION;INTELLECTUAL PROPERTY DEPARTMENT, 170 WOOD AVENUE SOUTH, ISELIN, NJ, 08830, US

(73) Assignee: Siemens Corporate Research, Inc., Princeton, NJ (US)

(21) Appl. No.: 11/934,253

(22) Filed: Nov. 2, 2007

Related U.S. Application Data
(60) Provisional application No. 60/915,461, filed on May 2, 2007.

(51) Int. Cl. A61B 5/02

(52) U.S. Cl. 600/500

(57) ABSTRACT

A method for measuring pulse wave velocity through an artery. The method includes: obtaining an image of the artery; generating from the image a model of the artery; selecting two reference points along the artery, such two reference points being separated a predetermined distance, D; observing a change in the model at a first one of the two reference points and a corresponding change in the model at a second one of the two reference points along with a time difference T between the observed changes; and estimating the pulse wave velocity, PWV, in accordance with PWV=D/T.

### CROSS-REFERENCE TO RELATED APPLICATIONS

This application claims priority from U.S. Provisional application No. 60/915,461 filed May 2, 2007, the entire subject matter thereof being incorporated herein by reference.

### TECHNICAL FIELD

This invention relates generally to methods for measuring pulse wave velocity and more particularly to methods for measuring pulse wave velocity using imaging.

### BACKGROUND

As is known in the art, pulse wave velocity (PWV) is a commonly used measure directly related to arterial distensibility (stiffness), thereby being an indicator for various cardiovascular diseases, including coronary artery disease, arteriosclerosis or connective tissue disorders.

During systole the contraction of the heart chambers create a pressure wave which travels down the arterial system. The speed of this pressure wave depends on the geometrical and elastic properties of the blood vessels, stiffer arterial walls result in faster wave propagation.

Current off-the-shelf PWV measurement systems involve applying two superficial sensors at proximal and distal locations of the arterial tree. They detect traversal times of the pulse wave mechanically (e.g. pressure sensors) or other sensing devices, e.g., (ultra-)sound. Although these methods yield an accurate measurement of the wave's transit time, the distance traveled can only be estimated by manual superficial measurement.

An MRI-based approach for measuring PWV was proposed by Gang G.; Mark, P.; Cockshott, P.; Foster, J.; Martin, T.; Blyth, K.; Steedman, T.; Elliott, A.; Dargie, H. & Groenning, B. Measurement of Pulse Wave Velocity using Magnetic Resonance Imaging Engineering in Medicine and Biology Society, 2004. IEMBS '04. 26th Annual International Conference of the IEEE, 2004, 2, 3684-3687. They suggest to measure pulse wave transit time based on short axis phase contrast MR images of the aorta. The vessel length (i.e. travel distance) is estimated from an anatomical long axis MR image. A significant amount of manual interaction is required in this approach, e.g. selecting the correct slice position and defining the region of interest (ROI).

### SUMMARY

In accordance with the present invention, a method is provided for measuring pulse wave velocity through an artery. The method includes: obtaining images of the artery; generating from the image a model of the artery; selecting two reference points along the artery, such two reference points being separated a predetermined distance, D; observing a change in the model at a first one of the two reference points and a corresponding change in the model at a second one of the two reference points along with a time difference T between the observed changes; and estimating the pulse wave velocity, PWV, in accordance with PWV=D/T.

It should be understood that since the upslope detection can be influenced by noise, change is also observed at various points in between the two reference points.

In one embodiment, the change is a sudden expansion of the artery along a path between the two reference points.

In one embodiment, the expansion is observed as an upslope in an artery diameter curve at each of the two reference points.

In one embodiment, the reference points are disposed along diameters of the artery.

In one embodiment, the reference points are disposed along the centerline of the artery.

In one embodiment, the onset of the radius increase in the artery is tracked using a quadratic sub-frame interpolation scheme.

In one embodiment, the distance is derived directly from the model by integrating the centerline length of the centerline of the artery between the two reference points.

With such method, no separate hardware is applied to the subject; the PWV measurement can potentially reach a higher accuracy due to the more accurate estimate of the distance traveled; it can allow a sector-wise vessel distensibility assessment instead of an average result, and possible sources of increased impedance (deformations, branching of arteries) can be seen immediately from a visualization of the model.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a flow chart of the process measuring pulse wave velocity through an artery according to the invention;

FIG. 2 is a diagram shows data flow in the real-time virtual\system,

FIG. 3(a) is a linked list structure to maintain the narrow band rows; FIG. 3(b) shows placement of the initial curve C0; and FIG. 3(c) shows extraction of the final curve.

Like reference symbols in the various drawings indicate like elements.

### DETAILED DESCRIPTION

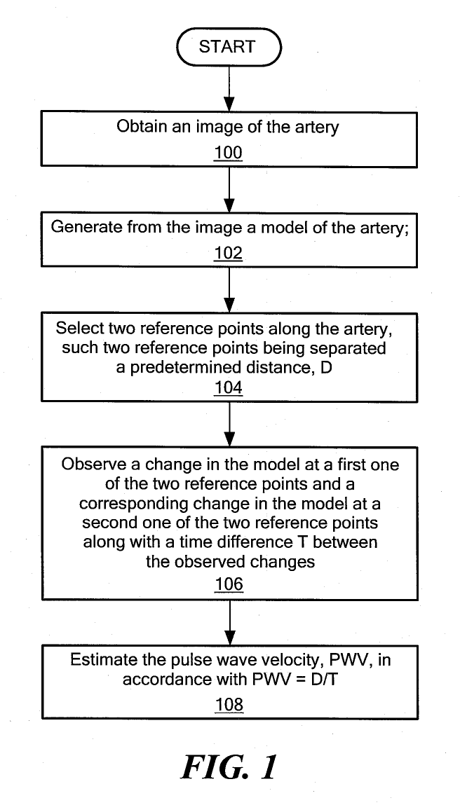

Referring now to FIG. 1, a flowchart is shown of the process used to for measuring pulse wave velocity through an artery. The method includes: obtaining images of the artery, Step 100; generating from the images a model of the artery, Step 102; selecting two reference points along the artery, such two reference points being separated a predetermined distance, D, Step 104; observing a change in the model at a first one of the two reference points and a corresponding change in the model at a second one of the two reference points along with a time difference T between the observed changes, Step 106; and estimating the pulse wave velocity, PWV, in accordance with PWV=D/T, Step 108. It should be understood that since the upslope detection can be influenced by noise, change is also observed at various points in between the two reference points.

The image of the artery may be obtained using MR, CT or ultrasound, for example. Here, in this example, MRI apparatus is used to obtain the image of a human heart, with the aorta artery thereof being segmented in 4D to thereby generate a model of the moving aorta.

As will be described in more detail below, the method for measuring pulse wave velocity (PWV), in contrast to conventional methods, relies purely on image data. No further hardware needs to be attached to the patient. In this method a 4D geometric model of the aorta is extracted from either a series of MR images/volumes, or
a stack of 2D cines, or
a series of images acquired automatically through interactive scanner control.

The basic model consists of a centerline defined by a series of node points along the aorta axis and a radius function at each of these discrete locations. An approximation of the true vessel lumen can be retrieved by interpolation between node points and radius values, e.g. by linear or B-Spline fitting.

Optionally a refining surface mesh can be available to take local deformations into account. The fourth dimension of the model represents the phase within the periodic cardiac cycle.

Finding points on the vessel wall is the first step to fit the model to the acquired data. Depending on the format of the data at hand this can be done automatically by a contour-finding segmentation algorithm. User interaction may be helpful to guide/correct the segmentation result.

After identifying a dense field of points on the vessel surface, an optimization algorithm can iteratively improve upon an initial set of model parameters to find the best fit with respect to a previously defined cost function. In general a higher density of surface points leads to a more detailed and accurate model. A more detailed description of a possible incarnation of the model and associated segmentation and optimization procedure can be found in Kirchberg, K. J.; Wimmer, A. & Lorenz, C. H. Modeling the Human Aorta for MR-Driven Real-Time Virtual Endoscopy MICCAI (1), 2006, 470-477 the entire subject matter thereof being incorporated herein by reference.

### Generation of Artery Model (Step 102)

For real-time interaction with the MR scanner we used a communication framework as described by Kirchberg, K. J., Wimmer, A., Lorenz, C. H.: Real-Time Virtual Endoscopy for MR-Guided Aortic Interventions. In: Proc. 14th ISMRM. (2006) 268 Binford, T.: Visual perception by computer. In: IEEE Conference on Systems and Control. (1971). It provides means of changing slice position and orientation during scanning as well as sending the resulting image data to the processing system.

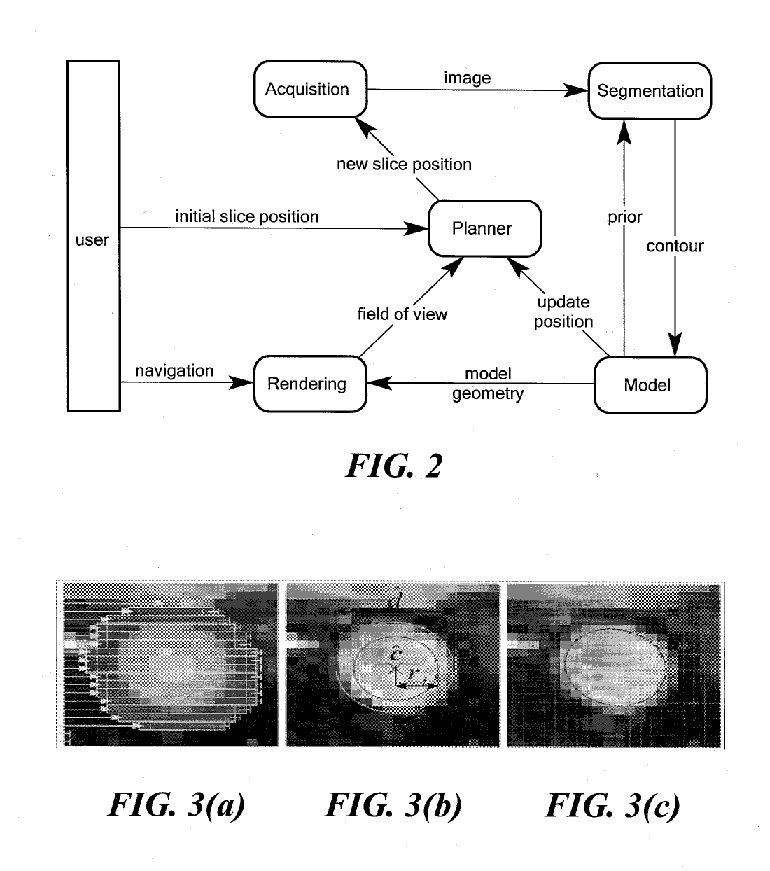

The planned workflow is as follows. Image slices are acquired perpendicular to the aorta's main axis. The scanned images are first subject to segmentation. Here, points on the aortic wall are extracted and their 3D position is determined by applying the (known) transformation of the image slice. After segmentation, the 3D coordinates of the contour points are added to the cloud of data points. An optimization is continuously fitting and updating the aorta model to best match the point cloud. The coherence information of contour points within one slice is not necessary in our approach, thus leading to a more general system that can easily be extended to other organs. Using the current model shape knowledge, new image slices are requested from the scanner. The layout of the system is depicted in FIG. 2 see also United States Patent Application 20060235671, entitled “Real-time virtual endoscopy”, published Oct. 19, 2006, inventors Kirchberg; Klaus J; et al., assigned to the same assignee as the present invention, the entire subject matter thereof being incorporated herein by reference.

Therefore, as a component of such a real time interventional system, we present methods for (1) optimized segmentation of the aorta and (2) a modified generalized cylinder (GC) model, both suitable for real time application. In particular, this work describes a GC model for the human aorta that is usable in the real-time MRI scenario. A novel reference frame scheme is introduced to solve the problem of singularities in the Frenet-Serret frame. The presented approach is kept general, making it easily adaptable to other organs.

We also present a highly optimized segmentation using Geodesic Active Contours suitable for real-time MRI.

### Segmentation of the Aortic Cross-Section

Oriented roughly perpendicular to the aorta's main axis, the image slices of the healthy aorta exhibit a more or less circular cross-section. In case of a pathology, e.g. an aneurysm, the contour can diverge even to a concave shape. To handle this variability, segmentation is performed using a level set based approach.

The interventional scenario imposes severe constraints on acquisition and processing time. Feedback of the intervention should be less than a second. To achieve reasonable update detail the system should be able to acquire and process at least 5 frames per second with an in-plane resolution of about 2 mm.

### The Segmentation Method

We segment the aortic cross-section using Geodesic Active Contours [see Caselles, V., Kimmel, R., Sapiro, G.: Geodesic active contours. International Journal of Computer Vision 22(1) (1997]. An initial curve C⁰ (see below) is placed in the image and deformed by a curvature dependent speed κ and a constant speed v in curve normal direction n according to equation (1). g is inversely related to the edge strength which is calculated from the input intensities l. It acts as a stopping function and slows down or stops the curve evolution at object boundaries. The second part of the right hand side in equation (1) constitutes an edge attraction speed in normal direction which increases the robustness for partially weak boundaries or boundaries with small gaps, a highly desirable property for images with reduced quality due to rapid acquisition times.

∂ₜC=g(κ+v)n−(∇g·n)n (1)

We use the Perona-Malik diffusivity [see Perona, P., Malik, J.: Scale-Space and Edge Detection Using Anisotropic Diffusion. IEEE Trans. Pattern Anal. Mach. Intell. 12(7) (1990] given by equation (2) as stopping function g. λ is a contrast parameter which controls the decrease of the stopping function with increasing edge strength. The gradient is approximated using the Sobel Operator.

$$g ( x ) = \frac{1}{1 + {( \frac{\vert ∇ I ( x ) \vert}{λ} )}^{2}} . \qquad ( 2 )$$

The curve evolution defined in equation (1) is tracked by the level set framework according to equation (3). The curve is embedded as the zero level set of the 2D level set function φ. In contrast to an explicit parametric representation, this implicit representation does not require any (re-)parameterizations during the evolution.

$${\partial }_{t} φ = \vert ∇ φ \vert ( div ( g \frac{∇ φ}{\vert ∇ φ \vert} ) + vg ) . \qquad ( 3 )$$

We use a semi-implicit numerical implementation of the level set PDE. This permits large time steps and thus only few updates of the level set function are required until the object boundaries are reached by the deforming curve.

A semi-implicit formulation usually comes with the burden of a high computational effort per update. In Weickert, J., Kuehne, G.: Fast methods for implicit active contour models. In Osher, S., Paragios, N., eds.: Geometric Level Set Methods in Imaging, Vision, and Graphics. Springer (2003), a discretization of equation (3) is proposed which allows the application of the Additive Operator Splitting technique [Weickert, J., ter Haar Romeny, B., Viergever, M.: Efficient and reliable schemes for nonlinear diffusion filtering. IEEE Trans. Imag. Proc. 7(3) (1998). Thereby the computational effort is drastically reduced, and an overall speedup of an order of magnitude compared to an explicit formulation of the level set PDE is reported.

The Narrow Band approach [O'Donnell, T., Boult, T., Fang, X. S., Gupta, A.: The extruded generalized cylinder: a deformable model for object recovery. In: CVPR. (1994) 174-181] is used to further increase the efficiency of the segmentation algorithm. The computational domain is restricted to grid points in a neighborhood around the zero level set, thus reducing the complexity from O(N²) to O(N) for a N×N grid. We use the linked list structure proposed in [Goldenberg, R., Kimmel, R., Rivlin, E., Rudzsky, M.: Fast geodesic active contours. IEEE Transactions on Image Processing 10(10) (2001) 1467-1475] to maintain which grid points are part of the narrow band. FIG. 3(a) shows the structure describing the narrow band rows. A modified Chamfer distance transformation [Krissian, K., Westin, C.: Fast and accurate redistancing for level set methods. Computer Aided Systems Theory (EUROCAST'03) (2003) 48-51] is used to re-initialize the narrow band once the zero level set comes close to its boundary.

### Placement of the Initial Curve

The initial curve C⁰ is placed within the cross-section and expanded in order to recover the boundary. Given estimates ĉ and \hat{d} on the center and approximate diameter of the cross-section from our model of the aorta, the initial curve is set to a circle around ĉ, as illustrated in FIG. 3(b). The radius rᵢ is set to a smaller value than half the diameter \hat{d} in order to ensure that the initial curve is within the cross-section also for inaccurate estimates.

### Extraction of the Final Curve

The level set function takes a steady state when the deforming curve has reached the object boundaries. The final curve is then extracted in sub-grid accuracy using the Marching Squares algorithm (FIG. 3(c)).

### Aorta Model

Geometric shape models and their application in medical image analysis have been studied extensively. A survey of methods can be found in [McInerney, T., Terzopoulos, D.: Deformable models in medical image analysis: a survey. Med Image Anal 1(2) (1996) 91-108].

A common deformable model for cylindrical structures is the generalized cylinder model [Binford, T.: Visual perception by computer. In: IEEE Conference on Systems and Control. (1971]. A generalized cylinder consists of a 3D space curve, called spine or centerline, and a variable 2D cross-section function. The modeled surface is then defined by sweeping the cross-section function along the spine using some sweep rules. However, this formulation has several weaknesses, e.g. the occurrence of discontinuities at certain points. To overcome those problems, several extensions have been proposed, e.g., [O'Donnell, T., Boult, T., Fang, X. S., Gupta, A.: The extruded generalized cylinder: a deformable model for object recovery. In: CVPR. (1994) 174-181, O'Donnell, T., Gupta, A., Boult, T. E.: A new model for the recovery of cylindrical structures from medical image data. In: CVRMed-MRCAS '97. (1997) 223-232].

We employ a circular cross-section function. This scheme models the healthy aorta as well as aortic abnormalities such as coarctation or aneurysm reasonably well while providing numerical stability for the model optimization process detailed below. In order to fit the model to the segmented surface points we define a cost functional that measures the quality of the fit. We then derive the Euler-Lagrange equation and perform the optimization by gradient descent.

Equation 4 depicts the cost functional, where c:[0,1] [symbol] R³ defines the centerline curve and r:[0,1] [symbol] R the radius function of the cross-section parametrized over the same interval. The function Γ_{σ}:R [symbol] R denotes the gaussian kernel weighting the influence of data points. Parameter ŝᵢ refers to the projection of data point pᵢ onto the centerline curve as shown below. The fixed start and end points of the curve are x₀ and x₁. The cost functional is regularized by the second and third term, weighted with factors λ and μ, respectively.

$$\begin{matrix}I ( c , r ) = {\int }_{0}^{1} \frac{1}{N} {\sum }_{i = 1}^{N} {Γ}_{σ} ( s - {\hat{s}}_{i} ) {( ‖ c ( s ) - {p}_{i} ‖ - r ( s ) )}^{2} d s + λ {\int }_{0}^{1} \frac{1}{2} {‖ ∇ c ( s ) ‖}^{2} d s + μ {\int }_{0}^{1} \frac{1}{2} {{r}^{′} ( s )}^{2} d s & ( 4 ) \\ c ( 0 ) = {x}_{0} , \\ c ( 1 ) = {x}_{1} , \\ {\hat{s}}_{i} = \text{arg} {min}_{s} ‖ c ( s ) - {p}_{i} ‖ & ( 5 )\end{matrix}$$

The corresponding Euler-Lagrange equations give rise to the gradient descent equations

$$\begin{matrix}\frac{\partial c}{\partial t} ( s ) = - \frac{1}{N} {\sum }_{i = 1}^{N} {Γ}_{σ} ( s - {\hat{s}}_{i} ) ( ‖ c ( s ) - {p}_{i} ‖ - r ( s ) ) \frac{c ( s ) - {p}_{i}}{‖ c ( s ) - {p}_{i} ‖} - {λΔ}_{s} c ( s ) \\ and & ( 6 ) \\ \frac{\partial r}{\partial t} ( s ) = - \frac{1}{N} {\sum }_{i = 1}^{N} {Γ}_{σ} ( s - {\hat{s}}_{i} ) ( ‖ c ( s ) - {p}_{i} ‖ - r ( s ) ) - μ \frac{{\partial }^{2}}{\partial {s}^{2}} r ( s ) . & ( 7 )\end{matrix}$$

### Defining a Reference Frame

When sweeping a 2D shape along a 3D space curve, one has to define the frame of reference, i.e. the local coordinate system for the cross-section function. This is important for building a 3D mesh and for generalizing the cross-section function.

A geometrically straightforward choice is the Frenet-Serret reference frame, as it was used in O'Donnell, T., Boult, T., Fang, X. S., Gupta, A.: The extruded generalized cylinder: a deformable model for object recovery. In: CVPR. (1994) 174-181. It defines the local coordinate system by the tangent, normal, and binormal vectors of the spine curve. Let again c denote the spine curve; we assume natural parametrization for simplicity. Then the basis vectors of the Frenet-Serret frame are defined as

$$Z = \frac{d c}{d s} , \\ X = \frac{\frac{d Z}{d s}}{‖ \frac{d Z}{d s} ‖} , and \\ Y = Z \times X . \qquad ( 8 )$$

As mentioned above, this formulation suffers from instabilities of the second derivative, which may lead to discontinuities. We employ here a less general, but more stable reference frame. We choose a fixed vector W that is known to be non-collinear to the spine curve at any point. This can be done by applying prior knowledge of the organ to be modeled; in our case we chose W to be the normal vector of the coronal plane, in which the aorta roughly stretches. The basis vectors are then defined by

$$Z = \frac{d c}{d s} , \\ X = Z \times W , and \\ Y = Z \times X . \qquad ( 9 )$$

Thus, a mathematical model of an organ or blood vessel after (or during) medical image acquisition can be advantageous for both qualitative and quantitative analysis of its function. When fitting a model to acquired (MR) data, i.e. finding the model parameters that best fit the data, all evaluation can be done on the model itself. No further reference to the original image data is necessary.

Selecting two reference points along the artery, such two reference points being separated a predetermined distance, D, Step 104; Observing a change in the model at a first one of the two reference points and a corresponding change in the model at a second one of the two reference points along with a time difference T between the observed changes, Step 106; and estimating the pulse wave velocity, PWV, in accordance with PWV=D/T, Step 108.

Once the model fitting procedure is completed and two reference points along the aorta are chosen, the PWV can be estimated from the model parameters without further analysis of the image data by

PWV=distance traveled/transit time. (1)

The pressure wave induces a sudden expansion of the artery along its path, which can be picked up as an upslope in the vessel diameter curve at each of the discrete centerline locations. The onset (local minimum) of the radius increase can be detected in these curves, each resulting in a crossing event (cₓ, c_{y}, c_{z}, cₜ). Due to the generally low temporal resolution of the input data the minima of the curve are detected using a quadratic sub-frame interpolation scheme.

Smoothness of this series of events can be used to eliminate outliers, i.e. positions for which the onset was not detected correctly. Finally, the time difference between upslope events at two reference points equals the pulse wave's transit time. The resulting PWV value can be used to estimate arterial distensibility in a conventional manner.

The two reference points for measuring passage of the pulse wave can be selected arbitrarily along the stretch of the model. However, for most accurate measurement it is advisable to place them as far apart as possible, i.e. proximal and distal ends of the vessel region covered by the model. Sector-wise assessment is obtained by placing the reference points at the two ends of the corresponding sector. Because all the analysis is performed after the model has been extracted, no further scanning of the subject is required.

The distance traveled can be derived directly from the model geometry by integrating the centerline length between the reference points. This approach yields a much more accurate result than manual superficial distance measurement as used in conventional PWV recording methods.

Finally PWV can be computed using equation (1).

The described method is not limited to MR images and can be applied to other imaging modalities, e.g. computed tomography (CT) scans or ultrasound imaging.

A number of embodiments of the invention have been described. Nevertheless, it will be understood that various modifications may be made without departing from the spirit and scope of the invention. Accordingly, other embodiments are within the scope of the following claims.

What is claimed is:

1. A method for measuring pulse wave velocity through an artery, comprising:
obtaining an image of the artery;
generating from the image a model of the artery;
selecting two reference points along the artery, such two reference points being separated a predetermined distance, D;
observing a change in the model at a first one of the two reference points and a corresponding change in the model at a second one of the two reference points along with a time difference T between the observed changes;
estimating the pulse wave velocity, PWV, in accordance with PWV=D/T.

2. The method recited in claim 1 wherein the change is a sudden expansion of the artery along a path between the two reference points.

3. The method recited in claim 2 wherein the expansion is observed as an upslope in an artery diameter curve at each of the two reference points.

4. The method recited in claim 3 wherein the reference points are disposed along diameters of the artery.

5. The method recited in claim 4 wherein the reference points are disposed along centerlines of the artery.

6. The method recited in claim 5 wherein the onset of the radius increase in the artery is tracked using a quadratic sub-frame interpolation scheme.

7. The method recited in claim 1 wherein the distance is derived directly from the model by integrating the centerline length of the centerline of the artery between the two reference points.
